(19) United States
(12) Patent Application Publication
(10) Pub. No.: US 2026/0207332 A1
(43) Pub. Date: Jul. 23, 2026

(54) Systems and Methods for Enhancing Access and Delivery for TAVR

(71) Applicant: St. Jude Medical, Cardiology Division, Inc., St. Paul, MN (US)

(72) Inventors: Peter J. Ness, Minneapolis, MN (US); Michael Shane Morrissey, St. Paul, MN (US); William H. Peckels, Houlton, WI (US)

(73) Assignee: St. Jude Medical, Cardiology Division, Inc., St. Paul, MN (US)

(21) Appl. No.: 19/444,815

(22) Filed: Jan. 9, 2026

Related U.S. Application Data
(60) Provisional application No. 63/748,133, filed on Jan. 22, 2025.

(51) Int. Cl. A61F 2/24; A61L 29/08; A61L 29/14

(52) U.S. Cl. CPC A61F 2/2433; A61L 29/08; A61L 29/14

(57) ABSTRACT

A system for performing a transcatheter heart valve replacement in a patient may include a delivery catheter having an inflatable balloon on a distal portion of the delivery catheter, and a distal tip at a distal end of the delivery catheter. A collapsible and expandable prosthetic heart valve is configured to be crimped on the balloon while the balloon is deflated in a delivery condition of the delivery catheter. An introducer has a proximal hub sized to remain outside of the patient, and a sheath extending distally from the proximal hub and sized to receive the delivery catheter therethrough while the delivery catheter is in the delivery condition. A lubricant is applied to one or more of (i) an outer surface of the delivery catheter, (ii) an outer surface of the prosthetic heart valve, or (iii) an inner surface of the introducer.

### CROSS-REFERENCE TO RELATED APPLICATIONS

This application claims priority to U.S. Provisional Patent Application No. 63/748,133, filed January 22, 2025, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

According to one aspect of the disclosure, a system for performing a transcatheter heart valve replacement in a patient includes a delivery catheter having an inflatable balloon on a distal portion of the delivery catheter, and a distal tip at a distal end of the delivery catheter, and a collapsible and expandable prosthetic heart valve configured to be crimped on the balloon while the balloon is deflated in a delivery condition of the delivery catheter. The system also includes an introducer having a proximal hub sized to remain outside of the patient, and a sheath extending distally from the proximal hub and sized to receive the delivery catheter therethrough while the delivery catheter is in the delivery condition. A lubricant is applied to one or more of (i) an outer surface of the delivery catheter, (ii) an outer surface of the prosthetic heart valve, or (iii) an inner surface of the introducer. The lubricant may be a glycerol-based lubricant. The lubricant may be a silicone-based lubricant. The lubricant may be applied to the outer surface of the prosthetic heart valve, the lubricant being applied to an outer cuff of the prosthetic heart valve, the outer cuff being oriented toward the distal tip of the delivery catheter when the delivery catheter is in the delivery condition. The lubricant may be applied to the outer surface of the delivery catheter, the lubricant being applied to the distal tip of the delivery catheter. In the delivery condition of the delivery catheter, the balloon may include a distal pillowed portion and a proximal pillowed portion, and the prosthetic heart valve may be crimped on the balloon between the distal pillowed portion and the proximal pillowed portion, and the lubricant may be applied to the outer surface of the delivery catheter, the lubricant being applied to the distal pillowed portion of the balloon. In the delivery condition of the delivery catheter, the balloon may include a distal pillowed portion and a proximal pillowed portion, and the prosthetic heart valve may be crimped on the balloon between the distal pillowed portion and the proximal pillowed portion, and the prosthetic heart valve may include an outer cuff oriented toward the distal tip of the delivery catheter when the delivery catheter is in the delivery condition, and the lubricant may not be applied to any portion of the prosthetic heart valve or the delivery catheter except for locations between the proximal end of the outer cuff and the distal end of the distal tip when the delivery catheter is in the delivery condition. The lubricant may be applied to an inner surface of the introducer, the lubricant being applied to an inner surface of the sheath. In the delivery condition of the delivery catheter, the balloon may include a distal pillowed portion and a proximal pillowed portion, and the prosthetic heart valve may be crimped on the balloon between the distal pillowed portion and the proximal pillowed portion, and the prosthetic heart valve may include an outer cuff oriented away from the distal tip of the delivery catheter when the delivery catheter is in the delivery condition, and the lubricant may not be applied to any portion of the prosthetic heart valve or the delivery catheter except for locations between the proximal end of the outer cuff and the distal end of the distal tip when the delivery catheter is in the delivery condition.

According to another aspect of the disclosure, a system for performing a transcatheter heart valve replacement in a patient includes a delivery catheter having an inflatable balloon on a distal portion of the delivery catheter, and a distal tip at a distal end of the delivery catheter, and a collapsible and expandable prosthetic heart valve configured to be crimped on the balloon while the balloon is deflated in a delivery condition of the delivery catheter. The system also includes an introducer having a proximal hub sized to remain outside of the patient, and a sheath extending distally from the proximal hub and sized to receive the delivery catheter therethrough while the delivery catheter is in the delivery condition. A pocket is formed on an interior portion of the introducer, and a lubricant is stored within the pocket so that, in an active condition of the introducer, the lubricant is exposed to (i) an interior volume of the proximal hub and/or (ii) an interior volume of the sheath. The pocket may be an annular recess. The introducer may be in the active condition at all times in which the lubricant is stored within the pocket. The system may also include a containment feature, wherein in an inactive condition of the introducer, the containment feature covers the pocket while the lubricant remains within the pocket to prevent the lubricant from being exposed to (i) the interior volume of the proximal hub and/or (ii) the interior volume of the sheath. The containment feature may be a film or sheet configured to be manually removed from the introducer to transition the introducer from the inactive condition to the active condition. The system may further include a loader tube, the loader tube being configured to cover the prosthetic heart valve and at least a portion of the balloon in the delivery condition of the delivery catheter. The loader tube may have an outer diameter so that, as the loader tube is advanced into the introducer while the delivery catheter is in the delivery condition, a distal end of the loader tube is configured to press against the containment feature to transition the introducer from the inactive condition to the inactive condition. The distal end of the loader tube may be porous. The distal end of the loader tube may include at least one axial slot. The distal end of the loader tube may be aligned with the pocket, and the lubricant within the pocket may be in fluidic communication with an interior volume of the loader tube. The loader tube may have an inner diameter, and the containment feature may have an inner diameter, the inner diameter of the loader tube being larger than the inner diameter of the containment feature.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the example of the handle of the delivery system shown in FIG. 4.

FIG. 6 is an enlarged view of an example of the distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the example of the inflation system of FIG. 8.

FIG. 10 is a perspective view of an example of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is a graph showing an example of insertion forces when advancing a delivery catheter through an introducer.

FIG. 13 is an enlarged view of an example of the distal end of the delivery system shown in FIG. 4 with added lubrication.

FIG. 14A is a schematic cross-section of a proximal hub of an introducer with a lubricant reservoir.

FIG. 14B is a schematic cross-section of the proximal hub of the introducer of FIG. 14A with the lubricant reservoir having a cover in a first position.

FIG. 14C is a schematic cross-section of the proximal hub of the introducer of FIG. 14B with the lubricant reservoir having a cover in a second position.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent’s interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (known as paravalvular or "PV" leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent’s interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (known as paravalvular or "PV" leakage).

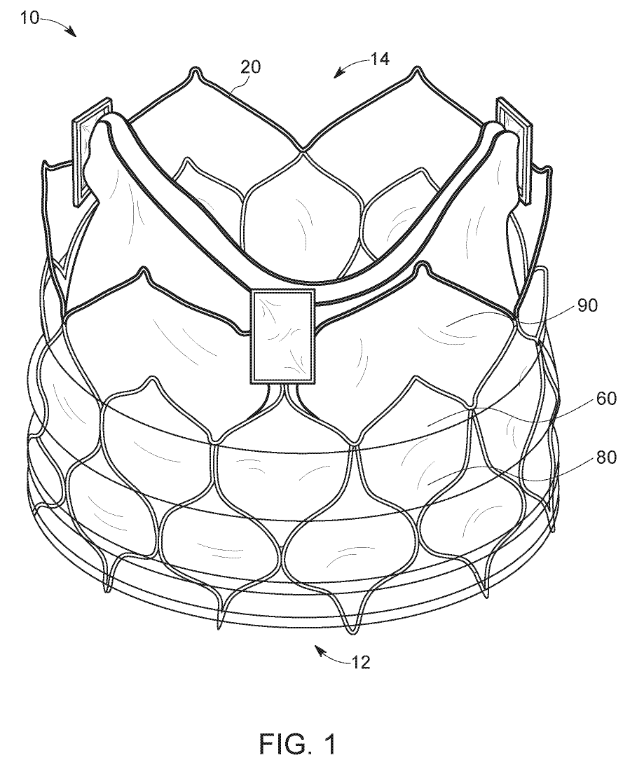

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

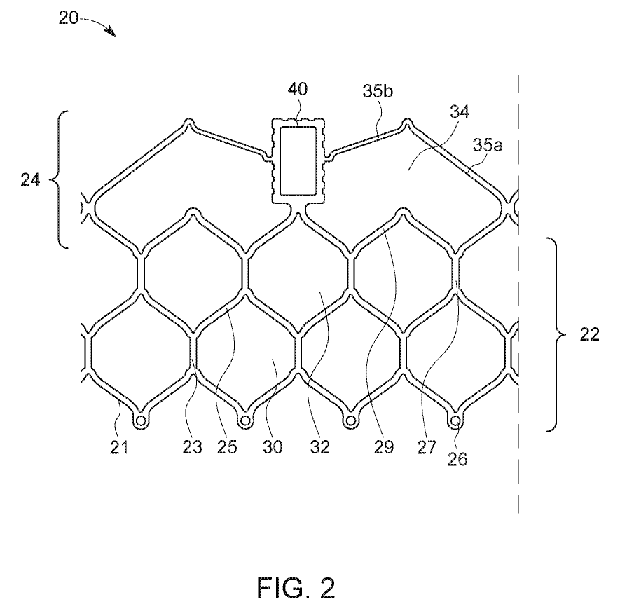

Now referring in addition to FIG. 2., FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed August 29, 2023 and titled “TAVI Deployment Accuracy - Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

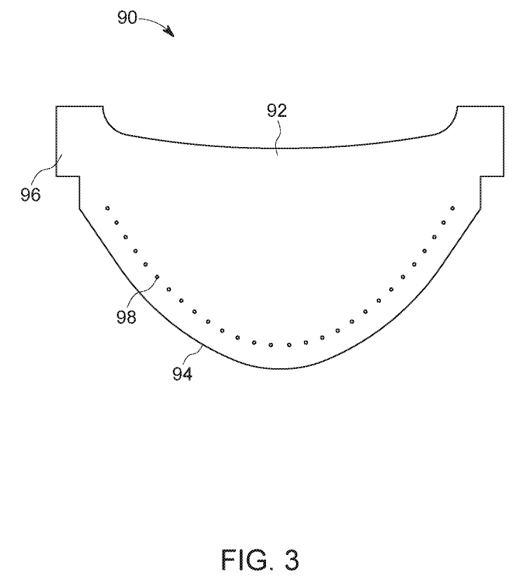

Now referring in addition to FIG. 3., FIG. 3 is a front view of an example of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

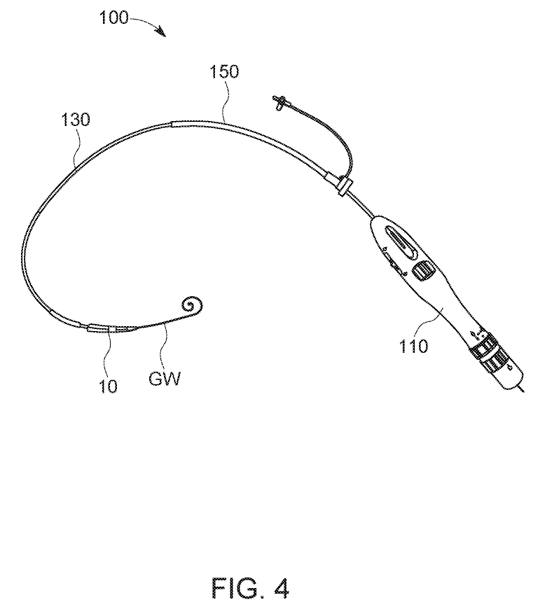

Now referring in addition to FIG. 4, FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introductory of 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient’s vasculature over the guidewire GW.

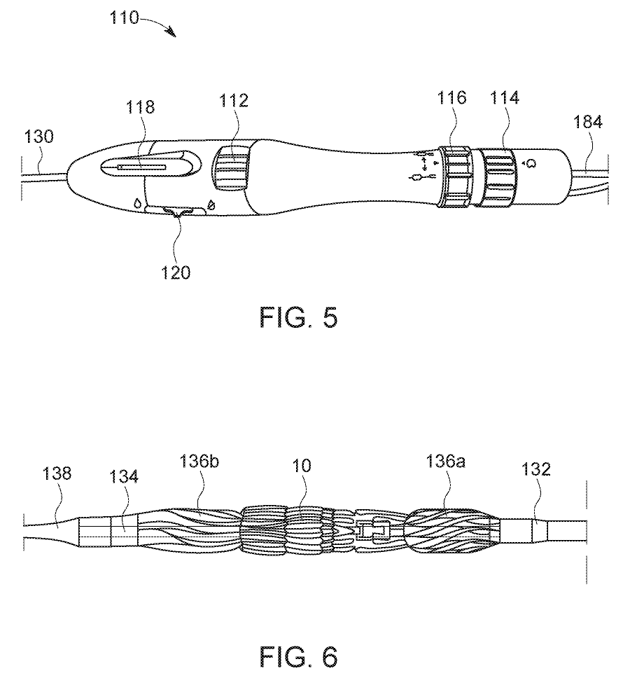

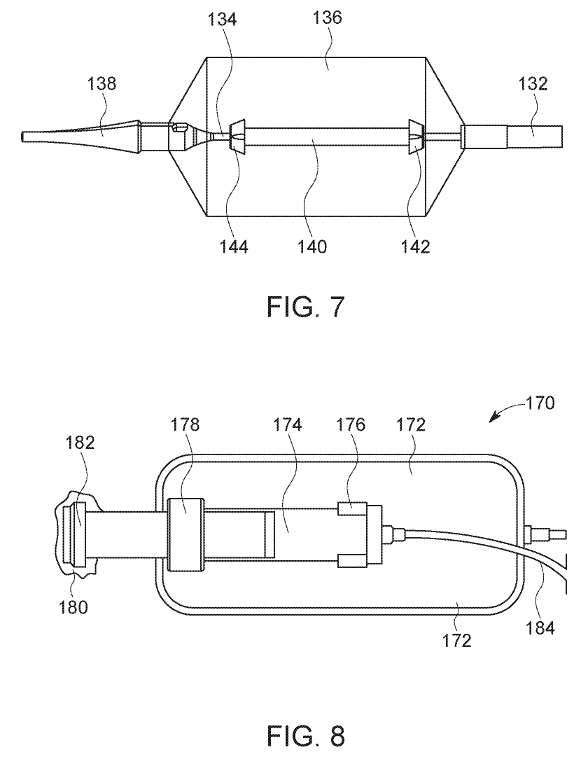

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g. the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/- 2.5 degrees of rotational alignment, within +/- 5 degrees of rotational alignment, within +/- 10 degrees of rotational alignment, within +/- 15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/- 0.5mm of axial alignment, within +/- 1.0mm of axial alignment, within +/- 1.5mm of axial alignment, within +/- 2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. Balloon actuator 120 may be an input device, for example similar to those described in connection with FIG. 16, that sends instructions to inflation system 170 or another device configured to control inflation. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed November 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient’s vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Now referring in addition to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

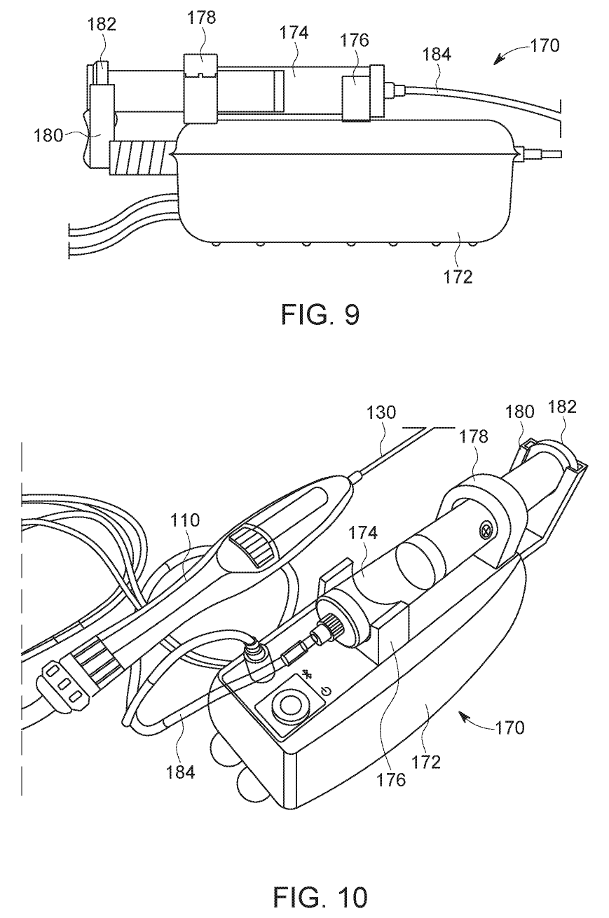

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. In some embodiments, the inflation system 170 forms a computer system, or components thereof, such as shown and described in connection with FIG. 16. Housing 172 may include one or more fixed cradles to receive a syringe 174. In the illustrated embodiment, a distal cradle 176 is provide with an open "C"- or "U"-shaped configuration so that the distal end of the syringe 174 may be snapped into or out of the distal cradle 176. A proximal cradle 178 may also be provided, which may have a "C"- or "U"-shaped bottom portion hingedly connected to a "C"- or "U"-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a "C"- or "U"-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a "U"-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in the examples of each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application No. 18/311,458, the disclosure of which is hereby incorporated by reference herein.

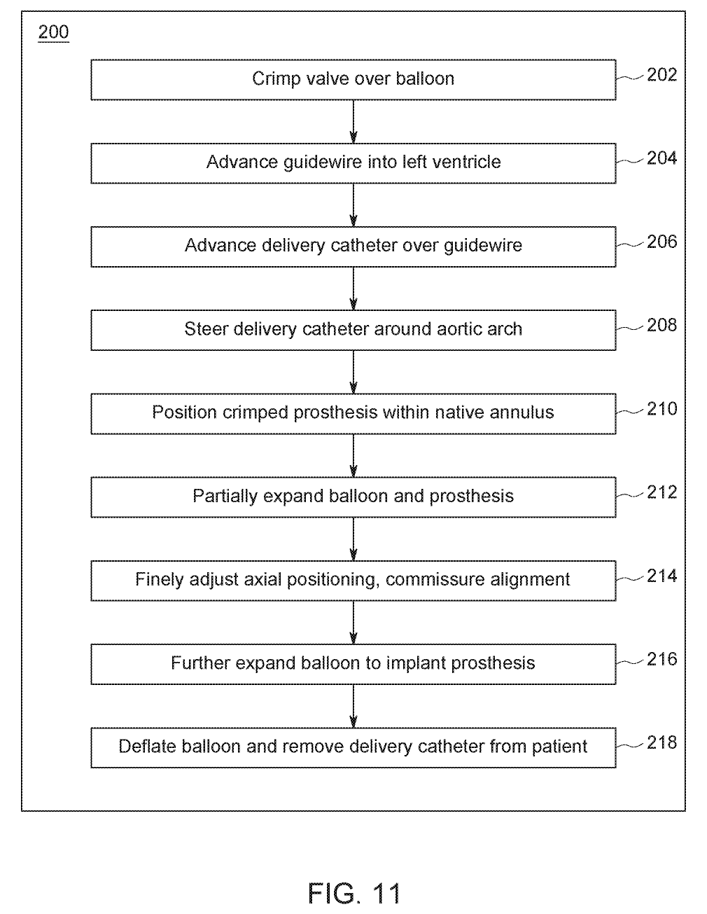

Now referring in addition to FIG. 11, FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient’s skin (or in contact with another device that enters the patient’s femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieve and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing balloon actuator 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

Referring briefly back to FIG. 4, in an example implantation of prosthetic heart valve 10, an introducer 150 (which may be an expandable introducer, and which may be a separate component that is not captured or pre-installed on the delivery catheter 130) is typically inserted into the patient’s vasculature, for example over a guidewire which has already been introduced into the vasculature. The introducer 150 may generally include a main introducer sheath which is configured to eventually receive therethrough the delivery catheter 130, and which may be sized to extend a distance into the vasculature. For example, in a transfemoral delivery of prosthetic heart valve 10, the main sheath may be inserted into the femoral artery until the main sheath extends between about 250mm and about 350mm into the femoral artery of the patient, with a proximal hub of the introducer 150 remaining outside of the patient. The proximal hub may include a hemostasis valve to help ensure that blood does not flow backwards through the introducer sheath. Once the introducer 150 is in place, the user may advance the delivery catheter 130 through the proximal hub of the introducer 150, across the hemostasis valve, and through the main introducer sheath until the distal end of the delivery catheter 130 exist the distal end of the main introducer sheath. After exiting the main introducer sheath, the use may continue to advance the prosthetic heart valve 10 on the delivery sheath 130 toward and around the aortic arch until the prosthetic heart valve 10 is positioned within the native valve annulus, at which point the prosthetic heart valve 10 may be deployed. In some examples, at least while the crimped prosthetic heart valve 10 is inserted through the hemostasis valve of the proximal hub of the introducer 150, a loader sheath may cover the prosthetic heart valve 10 to help assist passing the prosthetic heart valve 10 across the hemostasis valve without damaging the hemostasis valve or the prosthetic heart valve 10, and/or without causing the position of the prosthetic heart valve 10 to change relative to the balloon 136 onto which it is crimped. After the prosthetic heart valve 10 passes through the hemostasis valve, the loader sheath may be withdrawn or otherwise removed from the delivery catheter 130.

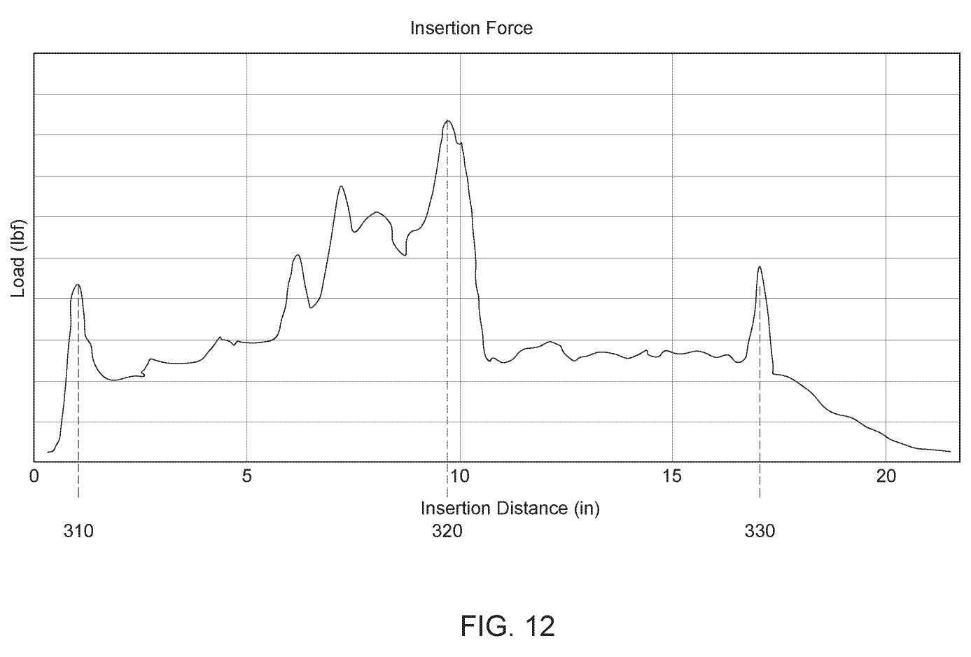

The process of advancing the prosthetic heart valve 10 and/or the delivery catheter 130 through the introducer 150 can be a manually difficult process, and even more so when using large diameter prosthetic heart valves and/or expandable introducer sheaths, which may generally create more friction and require more force to successfully pass the prosthetic heart valve 10 and/or the delivery catheter 130 through the introducer 150. For example, FIG. 12 illustrates an example graph showing insertion forces (shown as Load on the y-axis in pounds-force) as the delivery catheter 130 with prosthetic heart valve 10 crimped thereon passes a distance through the introducer 150 (shown as Insertion Distance on the x-axis in inches of distance through the introducer). In the particular graph illustrated in FIG. 12, each horizontal line in the chart may represent an increase in load by about 1 pound-force (“lbf”) or about 2 lbfs, with maximum loads during insertion being up to about 10 lbfs.

Referring to FIG. 12, a first characteristic spike 310 in insertion forces may be encountered as the prosthetic heart valve 10 and/or an overlying loader sheath pass through the hemostasis valve within the proximal hub of the introducer, which may be positioned outside of the patient. After the first spike 310, the insertion forces may tend to increase as the delivery catheter 130 and prosthetic heart valve 10 continue to be pushed distally through the introducer 150, until reaching a maximum insertion force, identified as 320 on the graph of FIG. 12. The maximum insertion force 130 may be reached, for example, when the proximal end of the expandable portion of the introducer sheath is first forced fully open. In other words, expandable introducer sheaths may have a proximal non-expandable section which transitions to a distal expandable section, and when the delivery system (e.g. the section of the delivery device carrying the crimped prosthetic heart valve) starts to pass the transition point and initially forces the beginning of the expandable section of the introducer sheath to maximally open, the insertion forces may be at a maximum. After the maximum insertion force is reached at 320, advancement of the delivery catheter 130 and the prosthetic heart valve 10 may continue, with the insertion forces being smaller than the maximum reached at 320. The insertion force may have another characteristic spike 330, which may be encountered as the prosthetic heart valve 10 itself exits the distal end of the introducer 150. Once the prosthetic heart valve 10 is clear of the distal end of the introducer 150, insertion forces may decrease and become generally insignificant compared to the forces encountered while the prosthetic heart valve 10 is being advanced through the introducer 150.

The example insertion forces required to advance the delivery catheter 130, and the prosthetic heart valve 10 mounted thereon, through the introducer 150 are not negligible. Rather, the insertion forces may create difficulty for the user in manually pushing the delivery catheter 130 and prosthetic heart valve 10 through the introducer 150. Further, not only do high insertion forces create ergonomic hindrances (e.g. fatigue or general difficulty) for the user, but high insertion forces may increase the likelihood that the prosthetic heart valve 10 becomes dislodged or otherwise unintentionally mispositioned relative to the balloon 136 on which the prosthetic heart valve 10 is mounted. Thus, it would be desirable to reduce the magnitude of insertion forces and/or reduce the variability of insertion forces and/or improve valve retention on the balloon when advancing the delivery catheter 130 and the prosthetic heart valve 10 through the introducer 150. Various examples described herein reduce the insertion forces (by reducing the magnitude of the insertion forces shown in FIG. 12) and/or reduce the variability of insertion forces (by reducing the differences between the peaks and the troughs of the insertion forces shown in FIG. 12) while advancing the delivery catheter 130 through the introducer 150, preferably without creating a significantly enhanced risk of the prosthetic heart valve 10 sliding relative to the balloon 136 during such advancement.

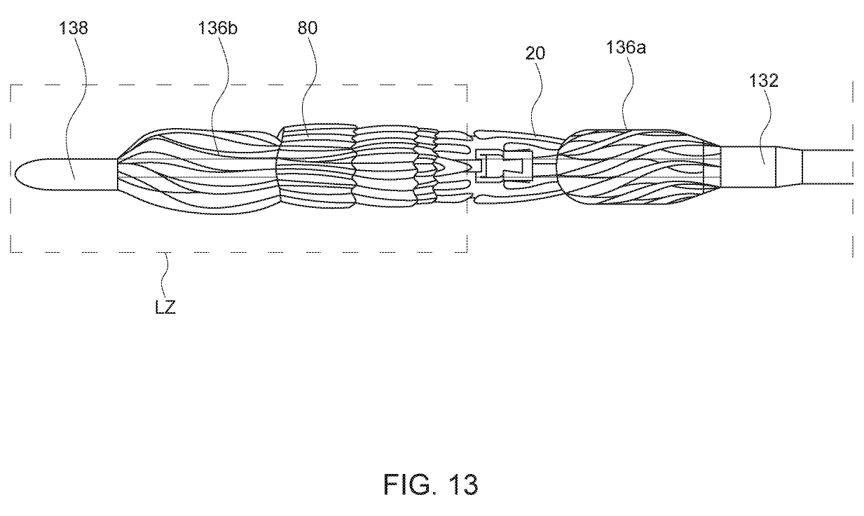

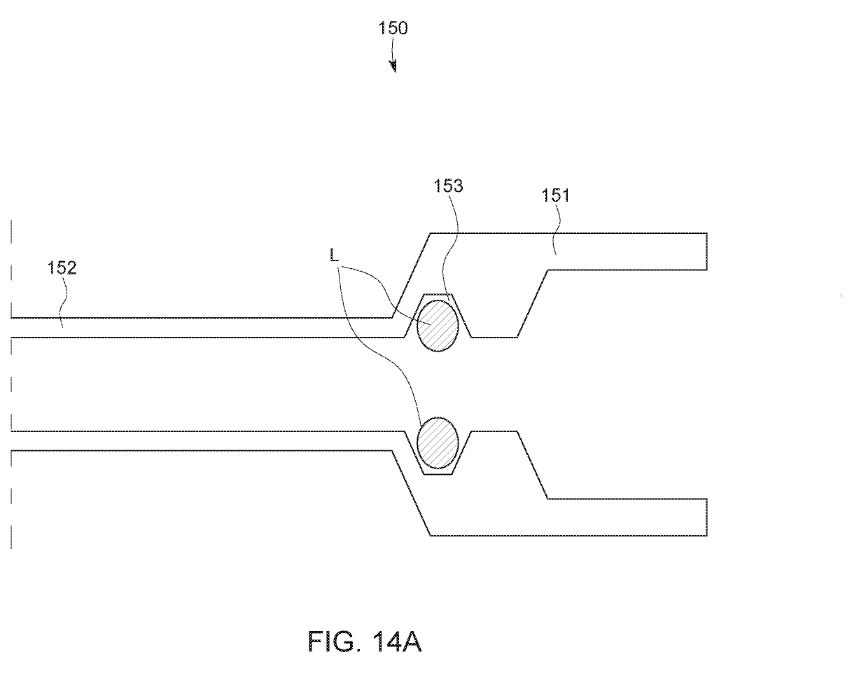

In one example, a lubricant may be provided on an outer surface of the delivery device 130 and/or prosthetic heart valve 10 that will come into contact with an inner surface of the main sheath of the introducer 150 (which may generally be limited to, although not necessarily only limited to, portions of the introducer distal of the hemostasis valve). FIG. 13 an enlarged view of the distal end of the delivery catheter 130 with the prosthetic heart valve 10 crimped over a balloon thereof, generally similar to the view shown in FIG. 6. For example, FIG. 13 shows a distal end portion of outer catheter 132 adjacent the balloon of the delivery catheter 132. As shown in FIG. 13, the balloon has been processed to include an enlarged proximal shoulder or pillow 136a and an enlarged distal shoulder or pillow 136b, with the prosthetic heart valve 10 being crimped over a central portion of the balloon between the proximal pillow 136a and the distal pillow 136b. The distal-most or leading end of the delivery catheter 130 may terminate in an atraumatic distal tip 138 as described above. In a transfemoral retrograde delivery of prosthetic heart valve 10, the inflow edge of the prosthetic heart valve 10 may be the leading edge (to the left in the view of FIG. 13). The outer skirt or cuff 80 may be the outermost portion of the prosthetic heart valve 10 that may contact the interior of the sheath of the introducer 150 for about half or more of the axial length of the crimped prosthetic heart valve 10. The inner cuff 60 and prosthetic leaflets 90 may generally be positioned radially inward of the frame 20 and thus be unlikely to be able to have meaningful contact with the inner surface of the main sheath of the introducer 150. Near the outflow end of the prosthetic heart valve 10 (to the right in the view of FIG. 13), the frame 20 may be the outermost component of the prosthetic heart valve 10 that is available to contact the inner surface of the main sheath of the introducer 150. On the delivery catheter 130 itself, the distal pillow 136b and proximal pillow 136a are likely to contact the inner surface of the main sheath of the introducer 150. The atraumatic distal tip 138, as the leading-most end of the delivery catheter 130, may also be likely to contact the inner surface of the main sheath of the introducer 150 during advancement.

In one example, friction between the outer surface of the prosthetic heart valve 10 and the inner surface of the introducer 150, and/or between the outer surface of component of the delivery device and the introducer 150, may be reduced using one or more lubricants. For example, one or more lubricants may be applied to the outer surface of the distal tip 138 of the delivery catheter 130 and/or to the outer surface of the balloon 136 on the delivery catheter 130. If lubricant is applied to the prosthetic heart valve 10 (in addition or instead of to components of the delivery device), the lubricant may be applied to the outer surface of the outer cuff 80 and/or the outer surface of the frame 20.

Although any biocompatible lubricant may be suitable for such application to the outer surface of the prosthetic heart valve 10 and/or to outer surfaces of components of the delivery device, particular examples include glycerol (or glycerol-based lubricants), silicone (or silicone-based lubricants), or other additives or compounds that have low coefficients of friction. Further various methods of actually applying the lubricant to the corresponding system component may be suitable, including for example via coating, infusion, or impregnation of the lubricant to the component.

In some examples, the lubricant may be applied throughout the length of the component, e.g. to both the proximal pillow 136a and distal pillow 136b, or to both the distal tip 138 and a length of the outer catheter 132 proximal to the proximal pillow 136a. However, in some examples the area of the system (e.g. the delivery device and/or the prosthetic heart valve 10 crimped thereon) to which lubricant is applied may be limited to a distal end of the system, including for example a lubrication zone LZ as shown in FIG. 13. In the particular example of FIG. 13, the lubrication zone LZ may be limited to any combination of, including all of, (i) the outer surface of the atraumatic tip 138, (ii) the outer surface of the distal pillow 136b, and (iii) the outer surface of the outer cuff 80. By limiting the application of lubricant(s) to a lubrication zone LZ at a distal or leading end of the system, as the delivery catheter 130 is advanced distally through the introducer 150, the lubricant may not only reduce friction when entering the introducer 150, but the lubricant may tend to disperse so that the lubricant reduces friction on proximal portions of the delivery device. For example, the lubricant may disperse onto the inner surface of the introducer sheath 150, reducing friction with trailing portions of the delivery catheter 130 as the delivery catheter 130 is advanced through the introducer sheath 150. Also, the lubricant may effectively flow proximally onto other proximal portions of the prosthetic heart valve 10 and/or onto other proximal portions of the delivery device as the delivery catheter 130 advances distally through the introducer 150. In other words, a targeted application of lubricant to a distal lubrication zone LZ may still provide friction reducing benefits to a portion of the delivery system (e.g. the prosthetic heart valve 10 and/or delivery device) proximal to the lubrication zone LZ. Although prosthetic heart valve 10 of FIG. 13 is a prosthetic aortic valve for transfemoral delivery, in other types of prosthetic heart valve deliveries (including prosthetic aortic valve deliveries using different routes than the transfemoral route, and other prosthetic heart valves such as prosthetic mitral and/or prosthetic tricuspid valves), the prosthetic heart valve may be in the reverse orientation relative to the delivery device compared to that shown in FIG. 13. For example, while the outer cuff 80 is oriented toward the distal tip 138, in other configurations, the outer cuff 80 (or a similar outer cuff, for example of a prosthetic mitral valve) may be oriented away from the distal tip 138. A person of skill in the art would understand that the concepts described in connection with FIG. 13 may similarly apply to prosthetic heart valves that may have a different orientation relative to the delivery system (e.g. instead of the inflow end of the prosthetic valve being at or oriented toward the leading end of the delivery system as in FIG. 13, the outflow end of the prosthetic valve may be at or oriented toward the leading end of the delivery system).

In some examples, if lubricant is applied to the outer surface of the balloon 136, it may be preferable to exclude the middle portion of the balloon 136 (e.g. between the distal pillow 136b and the proximal pillow 136a) from application of lubricant. Lubricating the middle portion of the balloon 136 may make it more likely that the prosthetic heart valve 10 might shift positions (e.g. slip along the length of the balloon 136) as the crimped prosthetic heart valve 10 is advanced through the introducer 150 and/or the vasculature. Friction between the prosthetic heart valve 10 and the center portion of the balloon 136 onto which it is crimped is an important factor in preventing the prosthetic heart valve 10 from undesirably slipping along the balloon 136. As the crimped prosthetic heart valve 10 is pushed through the introducer 150, contact between the outer surface of the prosthetic heart valve 10 and the inner surface of the introducer 150 creates friction (e.g. proximally directed force). If the proximally-directed frictional forces between the inner surface of the introducer 150 and the outer surface of the prosthetic heart valve 10 and are greater than the distally-directed frictional forces between the outer surface of the center portion of the balloon 136 and the inner surface of the prosthetic heart valve 10, the prosthetic heart valve 10 will slip proximally as it is pushed distally through the introducer 150. Thus, while application of lubricant may be desirable to reduce insertion forces (e.g. friction between the delivery catheter 130 and the introducer 150, and/or between the prosthetic heart valve 10 and the introducer 150), it is undesirable to significantly reduce valve retention force (e.g. friction between the prosthetic heart valve 10 and the middle portion of the balloon 136 on which it is crimped).

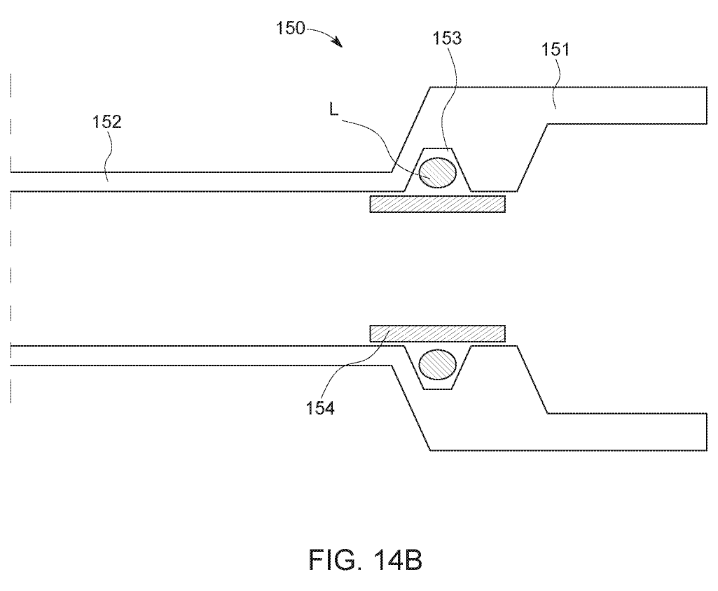

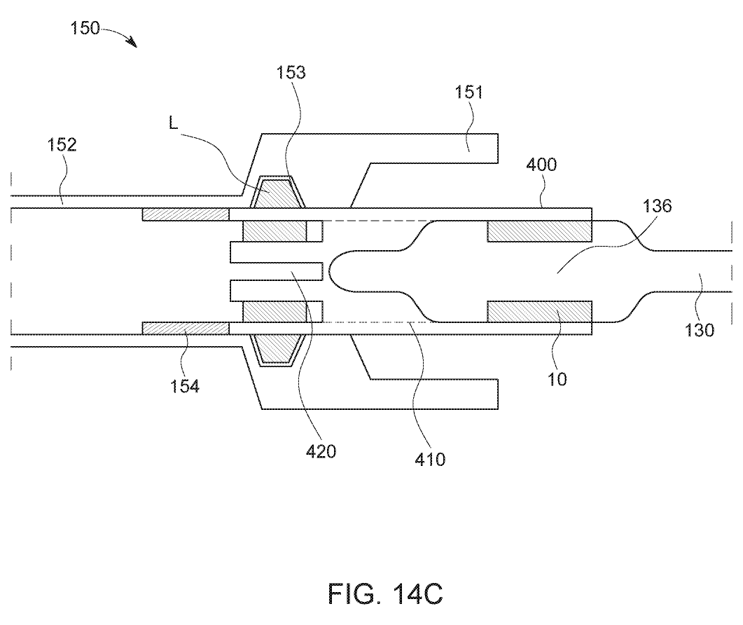

While some examples are provided above in which a lubricant is applied to the outer diameter of the prosthetic heart valve 10 and/or to the outer diameter of one or more components of the delivery device (e.g. delivery catheter 130 and/or the balloon 136 mounted thereon), in some examples either in addition to or as an alternative to such lubrication, a lubricant may be provided on an internal diameter of one or more components of the introducer 150. FIG. 14A illustrates a schematic cross-section a proximal end of introducer 150, including a proximal hub 151, which is configured to remain outside of the patient, and a main introducer sheath 152, which may be (but need not be) an expandable sheath, and which is configured to be positioned within the vasculature during the heart valve replacement procedure. Although not shown in FIG. 14A, a hemostasis valve may be positioned within introducer 150, for example within a portion of the proximal hub 151.

A lubricant, which may be similar to any of the lubricants described above, may be applied to one or more interior surfaces of the introducer 150, for example via impregnation, infusion coating, or any other suitable method of application. For example, any interior surface of the introducer 150 that is expected to come into contact with an outer surface of prosthetic heart valve 10 and/or components of the delivery device may have lubricant applied thereto. In some examples, this may include the interior of the proximal hub 151. However, as briefly described in greater detail below, the distal end of the delivery catheter 130 and the prosthetic heart valve 10 may be covered by another structure, which may be referred to as a loader tube 400, as those components pass through parts of the proximal hub 151.

Although lubricant may be applied to any portion of the inner surfaces of the introducer 150 to reduce friction when advancing the prosthetic heart valve 10 and the delivery device 130 through the introducer 150, in some examples the lubricant may be applied only to the proximal end portion of the inner surface of the main introducer sheath 152. Similar as is described above regarding the lubricant zone LZ of FIG. 13, if lubricant is applied only to the proximal end portion of the inner surface of the main introducer sheath 152, as the delivery catheter 130 is advanced distally through the introducer 150, the lubricant may not only reduce friction when entering the introducer sheath 152, but the lubricant may tend to disperse so that the lubricant reduces friction along a distal length of the introducer sheath 152. For example, the lubricant may disperse onto the outer surface of the prosthetic heart valve 10 and/or outer surfaces of one or more components of the delivery device. Also, the lubricant may effectively flow distally onto other distal portions of the introducer sheath 152 as the delivery catheter 130 advances distally through the introducer sheath 152. In other words, a targeted application of lubricant to a proximal end portion of the introducer sheath 152 may still provide friction reducing benefits along distal portions of the introducer sheath 152.

In some examples, either in addition to or as an alternative to applying lubricant directly to inner surfaces of the introducer 150, the introducer 150 may include a pocket 153 that may serve as a lubricant reservoir. In one example, shown in FIG. 14A, pocket 153 is formed as an annular recess extending circumferentially around an inner surface of the proximal hub 151. However, in other examples, the pocket 153 may take any form capable of acting as a reservoir, including as one or more individual recesses within the proximal hub 151. Prior to use of the introducer 150, the pocket 153 may be partially or completely filled with a lubricant L to form a lubricant reservoir. Lubricant L may be any of the lubricants described above, including for example glycerol-based or silicone-based lubricants. In some examples, the introducer 150 may be used in the condition shown in FIG. 14A, so that as the delivery catheter 130 and prosthetic heart valve 10 crimped thereon pass through the proximal hub 151, lubricant L contacts the outer surfaces of the delivery catheter 130 and/or prosthetic heart valve 10 to reduce friction during further advancement, and the lubricant L can disperse from the pocket 153 as the delivery device continues to advance relative to the pocket 153. It should be understood that, although the pocket 153 is described as being formed in the proximal hub 151, it may alternatively be formed in the main introducer sheath 152 or at a transition between the proximal hub 151 and the main introducer sheath 152.

In some examples, instead of leaving the pocket 153 open, the pocket 153 may be covered so that the lubricant L remains in the pocket 153 until the delivery device is advanced through the introducer 150. For example, FIG. 14B illustrates the introducer 150 of FIG. 14A with a containment feature 154 that covers the pocket 153 to maintain the lubricant L within the pocket 153 prior to use of the introducer 150. In some examples, the containment feature 154 may be a sleeve, a film, a sheet, a band, or another thin layer that, along with the portion of the proximal hub 151 forming the pocket 153, contains the lubricant L within the pocket 153. Just prior to use of the introducer 150, which may include before inserting the introducer 150 into the patient, or just prior to advancing the prosthetic heart valve 10 and the delivery catheter 130 through the introducer 130, the containment feature 154 may be manually removed. For example, the user may grasp the containment feature 154 manually, with forceps, or with a similar feature to peel away the containment feature 154 to expose the lubricant L.

In some examples, instead of manually removing the containment feature 154, the containment feature 154 may be configured to be removed (or to otherwise be triggered to expose the reservoir or lubricant L) upon the delivery device (or accessory components) passing into and/or through the introducer 150. For example, as noted above, and as shown in FIG. 14C, a loader tube 400 may be used to cover the distal end of the delivery catheter 130, including for example at least distal pillow 136b of balloon 130 and the prosthetic heart valve 10 mounted thereon. Loader tube 400 may be a rigid cylindrical member that assists the balloon 136 and the crimped prosthetic heart valve 10 in crossing the hemostasis valve (not shown) within the proximal hub 151 without damaging the balloon 136 or the prosthetic heart valve 10, while also avoiding causing the prosthetic heart valve 10 to shift position relative to the balloon 136. In other words, the loader tube 400 protects the prosthetic heart valve 10 as it traverses the hemostasis valve of the introducer 150. After fulfilling its function, the loader tube 400 may be left in place without further advancement, or may be removed from the introducer 150 by being withdrawn proximally over the delivery catheter 130, and in some examples can be peeled away and fully removed. Further details on loader tubes that may generally be suitable for use as loader tube 400 are described in U.S. Patent Application No. 18/943,056, filed November 11, 2024 and titled “Loader Sheath and Methods for Preparing Prosthetic Heart Valve Delivery System,” the disclosure of which is hereby incorporated by reference herein.

Still referring to FIG. 14C, loader tube 400 (which may also be referred to as a loader sheath) may be rigid and cylindrical. In some examples, although now shown in FIG. 14C, a proximal end of loader tube 400 may have a flange extending radially outwardly therefrom, limiting the ability of the loader tube 400 to pass too far into the main sheath 152 of the introducer 150. In the illustrated example, the loader tube 400 has a main body 410 with an outer diameter that is about equal to, or slightly smaller than, the inner diameter of the introducer 150 adjacent the pocket 153. With this configuration, as the loader sheath 400 covers the crimped prosthetic heart valve 10 and at least portions of the deflated balloon 136, as the loader tube 400 advances through the proximal hub 151, the distal end of the loader tube 400 pushes against, scrapes against, or otherwise contacts the containment feature 154 that covers the lubricant L. In some examples, the containment feature 154 is more substantial than a film, for example a cylindrical piece that is capable of sliding, but only a limited distance, relative to the pocket 153. For example, the containment feature 154 may be a cylinder with outer protrusions received in slots formed in the inner surface of the main sheath 152 distal to the pocket 153, so that as the distal end of the loader tube 400 is advanced while abutting a proximal end of the cylindrical containment feature 154, the containment feature 154 slides distally until the outer protrusions of the containment feature 154 reach the end of the slots in which they are sliding. In some examples, the inner diameter of the containment feature 154 may be sized slightly larger than the inner diameter of the loader tube 400 so as to not have a higher drag force. It should be understood that various other constructions may be suitable to allow for the containment feature 154 to be moved (including only a limited distance) by advancing the loader sheath 400 against the containment feature 154, to expose the pocket 153 containing the lubricant L.

In some examples, including that shown in FIG. 14C, the distal end of the loader tube 400 may be provided with features to help ensure that the lubricant L is able to readily move from the pocket 153 and into contact with outer surface of the prosthetic heart valve 10 and/or components of the delivery device (including the balloon 136 and/or the delivery catheter 130). For example, the embodiment shown in FIG. 14C includes a loader tube 400 having a porous distal tip created by a plurality of slots 420 alternating around the circumference of the distal end of the loader tube 400. With this example, and as shown in FIG. 14C, the structure of the distal end of the loader tube 400 that forms the slots 420 is strong enough to push the containment sheath 154 distally upon contact, while the lubricant L is able to pass through the slots 420 into contact with the outer surface of the prosthetic heart valve 10 and/or of components of the delivery device, such as the distal tip 138 or the balloon 136 (including the distal pillow 136b and/or proximal pillow 136a). With this configuration, the loader sheath 400 is advanced (while covering the prosthetic heart valve 10 and a distal portion of the delivery device) until the distal end of the loader tube 400 pushes aside the containment feature 154, exposing the interior of the pocket 153 housing the lubricant L. At this point, the loader sheath 400 may be held substantially static relative to the introducer 150, and the deliver catheter 130, along with the prosthetic heart valve 10 crimped on the balloon 136 thereof, is advanced beyond the loader tube 400 and into and through the main sheath 152. As the delivery device advances beyond the loader tube 400, the lubricant L is exposed to the delivery device through the slots 420 of the loader tube 400, allowing the lubricant L to disperse and reduce friction between (i) the outer surface of the prosthetic heart valve 10 and/or of the delivery device, and (ii) the inner surface of the introducer 150.

In some examples, when the lubricant L within the pocket 153 is exposed to the interior volume of the proximal hub 151 and/or sheath 152, such as in the example shown in FIG. 14A in which no containment feature 154 is provided, or such as in the example shown in FIG. 14C in which a containment features 154 is provided but has been moved or removed, the introducer 150 may be referred to as being in an active condition. On the other hand, when a containment feature 154 is provided and while that containment feature 154 covers the pocket 153 so that the lubricant L within the pocket 153 is not exposed to the interior volume of the proximal hub 151 and/or sheath 152, the introducer may be referred to as being in an inactive condition.

Whether lubricant is provided (i) on an outer surface of the prosthetic heart valve 10, (ii) on an outer surface of one or more components of the delivery device, including the distal tip 138 and/or the distal pillow 136b, (iii) on the inner surface of the main sheath 152 of the introduce 150, (iv) within the pocket 153 of the proximal hub 151 to form a reservoir of lubricant L, or any combination of (i) through (iv), the insertion forces of pushing the delivery catheter 130 may be reduced and/or have less variability during the delivery of the prosthetic heart valve 10 through the introducer 150, compared to a similar system without such lubrication. Although in some examples lubricant may be provided on large portions of the length of the introducer 150 and/or delivery catheter 130, as noted above, targeted application of lubricant over shorter distances may still provide desirable reduction in insertion forces, and targeted application may reduce overall volume of lubricant needed, which may help avoid requiring an increase in effective crimp profile of the prosthetic heart valve 10. Further, as noted above, although the lubricant desirably reduces insertion forces by reducing friction between the inner surface of the introducer 150 and the outer surfaces of the prosthetic heart valve 10 and/or delivery catheter 130 (or components thereof), the lubricant preferably does not significantly reduce friction between the prosthetic heart valve 10 and the balloon 136. In other words, as noted above, if the valve retention force is reduced too much due to lubricant, particularly to a point that is smaller than the valve insertion forces, the prosthetic heart valve 10 may become dislodged from the balloon 136 as the delivery catheter 130 is advanced through the introducer 150. Limited application of lubricant may help with avoiding an unintentionally large reduction in the valve retention forces.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for performing a transcatheter heart valve replacement in a patient, the system comprising:
a delivery catheter having an inflatable balloon on a distal portion of the delivery catheter, and a distal tip at a distal end of the delivery catheter;
a collapsible and expandable prosthetic heart valve configured to be crimped on the balloon while the balloon is deflated in a delivery condition of the delivery catheter; and
an introducer having a proximal hub sized to remain outside of the patient, and a sheath extending distally from the proximal hub and sized to receive the delivery catheter therethrough while the delivery catheter is in the delivery condition;
wherein a lubricant is applied to one or more of (i) an outer surface of the delivery catheter, (ii) an outer surface of the prosthetic heart valve, or (iii) an inner surface of the introducer.

2. The system of claim 1, wherein the lubricant is a glycerol-based lubricant.

3. The system of claim 1, wherein the lubricant is a silicone-based lubricant.

4. The system of claim 1, wherein the lubricant is applied to the outer surface of the prosthetic heart valve, the lubricant being applied to an outer cuff of the prosthetic heart valve, the outer cuff being oriented toward the distal tip of the delivery catheter when the delivery catheter is in the delivery condition.

5. The system of claim 1, wherein the lubricant is applied to the outer surface of the delivery catheter, the lubricant being applied to the distal tip of the delivery catheter.

6. The system of claim 1, wherein, in the delivery condition of the delivery catheter, the balloon includes a distal pillowed portion and a proximal pillowed portion, and the prosthetic heart valve is crimped on the balloon between the distal pillowed portion and the proximal pillowed portion, wherein the lubricant is applied to the outer surface of the delivery catheter, the lubricant being applied to the distal pillowed portion of the balloon.

7. The system of claim 1, wherein, in the delivery condition of the delivery catheter, the balloon includes a distal pillowed portion and a proximal pillowed portion, and the prosthetic heart valve is crimped on the balloon between the distal pillowed portion and the proximal pillowed portion, and the prosthetic heart valve includes an outer cuff oriented toward the distal tip of the delivery catheter when the delivery catheter is in the delivery condition, wherein the lubricant is not applied to any portion of the prosthetic heart valve or the delivery catheter except for locations between the proximal end of the outer cuff and the distal end of the distal tip when the delivery catheter is in the delivery condition.

8. The system of claim 1, wherein the lubricant is applied to an inner surface of the introducer, the lubricant being applied to an inner surface of the sheath.

9. The system of claim 1, wherein, in the delivery condition of the delivery catheter, the balloon includes a distal pillowed portion and a proximal pillowed portion, and the prosthetic heart valve is crimped on the balloon between the distal pillowed portion and the proximal pillowed portion, and the prosthetic heart valve includes an outer cuff oriented away from the distal tip of the delivery catheter when the delivery catheter is in the delivery condition, wherein the lubricant is not applied to any portion of the prosthetic heart valve or the delivery catheter except for locations between the proximal end of the outer cuff and the distal end of the distal tip when the delivery catheter is in the delivery condition.

10. A system for performing a transcatheter heart valve replacement in a patient, the system comprising:
a delivery catheter having an inflatable balloon on a distal portion of the delivery catheter, and a distal tip at a distal end of the delivery catheter;
a collapsible and expandable prosthetic heart valve configured to be crimped on the balloon while the balloon is deflated in a delivery condition of the delivery catheter; and
an introducer having a proximal hub sized to remain outside of the patient, and a sheath extending distally from the proximal hub and sized to receive the delivery catheter therethrough while the delivery catheter is in the delivery condition;
wherein a pocket is formed on an interior portion of the introducer, and a lubricant is stored within the pocket so that, in an active condition of the introducer, the lubricant is exposed to (i) an interior volume of the proximal hub and/or (ii) an interior volume of the sheath.

11. The system of claim 10, wherein the pocket is an annular recess.

12. The system of claim 10, wherein the introducer is in the active condition at all times in which the lubricant is stored within the pocket.

13. The system of claim 10, further comprising a containment feature, wherein in an inactive condition of the introducer, the containment feature covers the pocket while the lubricant remains within the pocket to prevent the lubricant from being exposed to (i) the interior volume of the proximal hub and/or (ii) the interior volume of the sheath.

14. The system of claim 13, wherein the containment feature is a film or sheet configured to be manually removed from the introducer to transition the introducer from the inactive condition to the active condition.

15. The system of claim 13, wherein the system further comprises a loader tube, the loader tube being configured to cover the prosthetic heart valve and at least a portion of the balloon in the delivery condition of the delivery catheter.

16. The system of claim 15, wherein the loader tube has an outer diameter so that, as the loader tube is advanced into the introducer while the delivery catheter is in the delivery condition, a distal end of the loader tube is configured to press against the containment feature to transition the introducer from the inactive condition to the inactive condition.

17. The system of claim 16, wherein the distal end of the loader tube is porous.

18. The system of claim 16, wherein the distal end of the loader tube includes at least one axial slot.

19. The system of claim 16, wherein when the distal end of the loader tube is aligned with the pocket, the lubricant within the pocket is in fluidic communication with an interior volume of the loader tube.

20. The system of claim 15, wherein the loader tube has an inner diameter, and the containment feature has an inner diameter, the inner diameter of the loader tube being larger than the inner diameter of the containment feature.
